(19) United States
(12) Patent Application Publication
(10) Pub. No.: US 2018/0000711 A1
(43) Pub. Date: Jan. 4, 2018

(54) POTASSIUM-CONTAINING AMPHOACETATE AND BETAINE SURFACTANTS

(71) Applicant: RHODIA OPERATIONS, Paris (FR)

(72) Inventors: Stewart Alexander WARBURTON, West Windsor, NJ (US); Paul HUBER, Buford, GA (US); David Michael RAZMUS, Trenton, NJ (US); Juie TANAVADE, Hillsborough, NJ (US)

(73) Assignee: RHODIA OPERATIONS, Paris (FR)

(21) Appl. No.: 15/637,063

(22) Filed: Jun. 29, 2017

Related U.S. Application Data
(60) Provisional application No. 62/356,706, filed on Jun. 30, 2016.

(51) Int. Cl. A61K 8/49; A61K 8/34; A61K 8/04; A61K 8/42; A61Q 19/10; A61K 8/46

(52) U.S. Cl. CPC A61K 8/494; A61Q 19/10; A61K 8/463; A61K 8/046; A61K 8/42; A61K 8/34; A61K 2800/52

(57) ABSTRACT

Compositions that include at least one potassium-containing amphoacetate and/or betaine surfactant, water and at least one additional surfactant selected from anionic surfactants, cationic surfactants, non-ionic surfactants, zwitterionic surfactants, and mixtures thereof.

### CROSS-REFERENCE TO RELATED APPLICATION

The present application claims the benefit of priority under 35 U.S.C. §119(e) of U.S. Provisional Application Ser. No. 62/356,706, filed on Jun. 30, 2016, the entire disclosure of which is incorporated herein by reference.

### BACKGROUND

Sustainability initiatives have led to an increased demand for concentrated compositions for personal, home and industrial use. Such products are popular due to their reduced impact on the environment. For example, concentrated compositions require less packaging, which cuts down on the amount of plastic and paper used. Additionally, concentrated compositions are formulated with less water, which reduces shipping costs for the materials.

Amphoteric surfactant compositions are useful in personal care applications, such as shampoos, body wash, hand soap, lotions, creams, conditioners, shaving products, facial washes, neutralizing shampoos, and skin treatments, in home care and industrial cleaning applications, such as liquid detergents, laundry detergents, hard surface cleansers, dish wash liquids, toilet bowl cleaners, and in other applications, such as oil field applications.

A need exists for sustainable amphoteric surfactant compositions for personal, home and industrial use that can be prepared at higher surfactant concentrations in comparison with conventional surfactants.

### SUMMARY

The present disclosure provides compositions that include at least one potassium-containing amphoacetate and/or betaine surfactant, water and at least one additional surfactant selected from anionic surfactants, cationic surfactants, non-ionic surfactants, zwitterionic surfactants, and mixtures thereof. In an embodiment, the composition includes at least one potassium-containing amphoacetate selected from amphoacetates according to Formulas I, II, III, IV, and combinations thereof. In another embodiment, the composition includes at least one potassium-containing betaine that includes potassium chloride and a betaine selected from coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and combinations thereof.

### DETAILED DESCRIPTION

Compositions according to the present disclosure include a potassium-containing amphoacetate and/or betaine surfactant. Such compositions provide enhanced benefits including increased concentration of surfactant loading and/or retaining flow at lower temperatures compared to compositions comprising none of these potassium-containing surfactants. In an embodiment, the composition includes a potassium-containing amphoacetate selected from: amphoacetates according to Formula I:

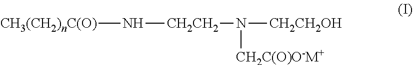

wherein n is from 6 to 20 and M is a potassium cation;

amphodiacetates according to Formula II:

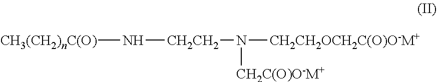

wherein n is from 6 to 20 and M is a potassium cation;

amphodiacetates according to Formula III:

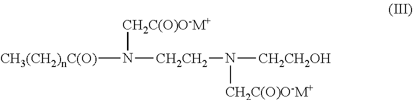

wherein n is from 6 to 20 and M is a potassium cation; and

amphodiacetates according to Formula IV:

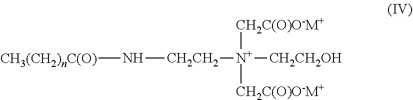

wherein n is from 6 to 20 and M is a potassium cation; and combinations thereof.

In another embodiment, the potassium-containing betaine surfactant composition includes a betaine and potassium chloride. In an embodiment, the betaine is selected from coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The term “betaine” also includes sulfobetaines. In an embodiment, the composition includes a sulfobetaine selected from coco dimethyl sulfopropyl betaine, stearyl dimethyl sulfopropyl betaine, lauryl dimethyl sulfoethyl betaine, lauryl bis-(2-hydroxyethyl) sulfopropyl betaine, and combinations thereof. In another embodiment, the composition includes a betaine selected from amidobetaines and amidosulfobetaines, wherein the RCONH(CH₂)₃ radical is attached to the nitrogen atom of the betaine.

In an embodiment, the potassium chloride salt is present in the composition at a weight concentration of from about 5% to about 12%.

In one embodiment, compositions according to the present disclosure exhibit one or more lamellar surfactant phases. “Lamellar surfactant phases” are phases which include one or more surfactant bilayers, typically a plurality of surfactant bilayers separated by liquid medium. Lamellar phases include spherulite phases and the typical form of the liquid crystal G-phase, as well as mixtures thereof “G-phases”, which are sometimes referred to in the literature as “L, phases”, are typically pourable, non-Newtonian, anisotropic products that are cloudy looking and exhibit a characteristic “smeary” appearance on flowing. Lamellar phases can exist in several different forms, including domains of parallel sheets, which constitute the bulk of the typical G-phases described above.

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

Applications

The potassium-containing amphoacetate and/or betaine surfactants of the present disclosure are useful in, for example, personal care applications, such as shampoos, bath and shower products (body cleansers, body wash, shower gel, liquid soap, soap bars, syndet bars, conditioning liquid bath oil, bubble bath, bath powders, and the like), hand soap, facial washes, neutralizing shampoos, and personal wipes, and in home care and industrial cleaning applications, such as liquid detergents, laundry detergents, hard surface cleansers, dish wash liquids, toilet bowl cleaners, as well as other applications, such as oil field applications.

Personal Care

In one embodiment, the present disclosure is directed to a personal care composition that includes one or more potassium-containing amphoacetate and/or betaine surfactants, water, and one or more additional components as described herein.

In an embodiment, the one or more additional components are selected from one or more additional surfactants, one or more additional benefit agents, such as emollients, moisturizers, conditioners, skin conditioners, hair conditioners, vitamins or their derivatives, antioxidants, free-radical scavengers, abrasives, anti-UV agents, UV absorbers, antimicrobial agents, antibacterial agents, antifungal agents, anti-acne agents, antiseborrhoeic agents, anti-ageing agents, anti-wrinkle agents, anti-inflammatory agents, refreshing agents, cicatrizing agents, vascular-protection agents, antiperspirants, deodorants, immunomodulators, nourishing agents, essential oils, fragrances, and combinations thereof.

Suitable additional surfactants include anionic surfactants, cationic surfactants, non-ionic surfactants, zwitterionic surfactants, and mixtures thereof. As used herein, the phrases “additional surfactant” and “additional surfactants” refer to surfactants different from the potassium-containing amphoacetate and betaine surfactants.

Suitable anionic surfactants are known compounds and include, for example, linear alkylbenzene sulfonates, alpha olefin sulfonates, paraffin sulfonates, alkyl ester sulfonates, alkyl sulfates, alkyl alkoxy sulfates, alkyl sulfonates, alkyl alkoxy carboxylates, alkyl alkoxylated sulfates, monoalkyl phosphates, dialkyl phosphates, sarcosinates, isethionates, and taurates, as well as mixtures thereof, such as for example, ammonium lauryl sulfate, ammonium laureth sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium trideceth sulfate, sodium tridecyl sulfate, ammonium trideceth sulfate, ammonium tridecyl sulfate, sodium cocoyl isethionate, disodium laureth sulfosuccinate, sodium methyl oleoyl taurate, sodium laureth carboxylate, sodium trideceth carboxylate, sodium monoalkyl phosphate, sodium dialkyl phosphate, sodium lauryl sarcosinate, lauroyl sarcosine, cocoyl sarcosinate, ammonium cocyl sulfate, sodium cocyl sulfate, potassium cocyl sulfate, monoethanolamine cocyl sulfate, sodium tridecyl benzene sulfonate, sodium dodecyl benzene sulfonate, glycinates, such as for example, sodium lauroyl glycinate, sodium cocoyl glycinate, glutamates, such as for example, sodium cocoyl glutamate, and mixtures thereof.

The cationic counterion of the anionic surfactant is typically a sodium cation but may alternatively be a potassium, lithium, calcium, magnesium, ammonium cation, or an alkyl ammonium anion having up to 6 aliphatic carbon atoms, such as anisopropylammonium, monoethanolammonium, diethanolammonium, or triethanolammonium cation. Ammonium and ethanolammonium salts are generally more soluble than the sodium salts. Mixtures of the above cations may be used.

Suitable cationic surfactants are known compounds and include, for example, mono-cationic surfactants according to Formula (V) below:

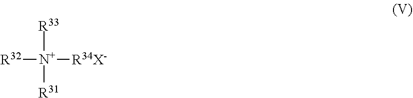

wherein R³¹, R³², R³³ and R³⁴ are independently hydrogen or an organic group, provided that at least one of R³¹, R³², R³³ and R³⁴ is not hydrogen, and X⁻ is an anion, as well as mixtures of such compounds.

If one to three of the R³¹, R³², R³³ and R³⁴ groups are each hydrogen, then the compound may be referred to as an amine salt. Some examples of cationic amine salts include polyethoxylated (2) oleyl/stearyl amine, ethoxylated tallow amine, cocoalkylamine, oleylamine, and tallow alkyl amine.

For quaternary ammonium compounds (generally referred to as quats) R³¹, R³², R³³ and R³⁴ may be the same or different organic group, but may not be hydrogen. In one embodiment, R³¹, R³², R³³ and R³⁴ are each C8-C24 branched or linear hydrocarbon groups which may comprise additional functionality such as, for example, fatty acids or derivatives thereof, including esters of fatty acids and fatty acids with alkoxylated groups; alkyl amido groups; aromatic rings; heterocyclic rings; phosphate groups; epoxy groups; and hydroxyl groups. The nitrogen atom may also be part of a heterocyclic or aromatic ring system, e.g., cetethyl morpholinium ethosulfate or steapyrium chloride.

Examples of quaternary ammonium compounds of the monoalkyl amine derivative type include: cetyl trimethyl ammonium bromide (also known as CETAB or cetrimonium bromide), cetyl trimethyl ammonium chloride (also known as cetrimonium chloride), myristyl trimethyl ammonium bromide (also known as myrtrimonium bromide or Quaternium-13), stearyl dimethyl benzyl ammonium chloride (also known as stearalkonium chloride), oleyl dimethyl benzyl ammonium chloride, (also known as olealkonium chloride), lauryl/myristryl trimethyl ammonium methosulfate (also known as cocotrimonium methosulfate), cetyl dimethyl (2)hydroxyethyl ammonium dihydrogen phosphate (also known as hydroxyethyl cetyldimonium phosphate), babassuamidopropalkonium chloride, cocotrimonium chloride, distearyldimonium chloride, wheat germ-amidopropalkonium chloride, stearyl octyldimonium methosulfate, isostearaminopropalkonium chloride, dihydroxypropyl PEG-5 linoleaminium chloride, PEG-2 stearmonium chloride, Quaternium 18, Quaternium 80, Quaternium 82, Quaternium 84, behentrimonium chloride, dicetyl dimonium chloride, behentrimonium methosulfate, tallow trimonium chloride and behenamidopropyl ethyl dimonium ethosulfate.

Quaternary ammonium compounds of the dialkyl amine derivative type include, for example, distearyldimonium chloride, dicetyl dimonium chloride, stearyl octyldimonium methosulfate, dihydrogenated palmoylethyl hydroxyethylmonium methosulfate, dipalmitoylethyl hydroxyethylmonium methosulfate, dioleoylethyl hydroxyethylmonium methosulfate, hydroxypropyl bisstearyldimonium chloride, and mixtures thereof.

Quaternary ammonium compounds of the imidazoline derivative type include, for example, isostearyl benzylimidonium chloride, cocoyl benzyl hydroxyethyl imidazolinium chloride, cocoyl hydroxyethylimidazolinium PG-chloride phosphate, Quaternium 32, and stearyl hydroxyethylimidonium chloride, and mixtures thereof.

Typical cationic surfactants comprise dialkyl derivatives such as dicetyl dimonium chloride and distearyldimonium chloride; branched and/or unsaturated cationic surfactants such as isostearylaminopropalkonium chloride or olealkonium chloride; long chain cationic surfactants such as stearalkonium chloride and behentrimonium chloride; as well as mixtures thereof.

Suitable anionic counterions for the cationic surfactant include, for example, chloride, bromide, methosulfate, ethosulfate, lactate, saccharinate, acetate and phosphate anions.

Suitable nonionic surfactants are known compounds and include amine oxides, fatty alcohols, alkoxylated alcohols, fatty acids, fatty acid esters, and alkanolamides. Suitable amine oxides comprise, (C10-C24) saturated or unsaturated branched or straight chain alkyl dimethyl oxides or alkyl amidopropyl amine oxides, such as for example, lauramine oxide, cocamine oxide, stearamine oxide, stearamidopropylamine oxide, palmitamidopropylamine oxide, decylamine oxide as well as mixtures thereof. Suitable fatty alcohols include, for example, (C10-C24) saturated or unsaturated branched or straight chain alcohols, more typically (C10-C20) saturated or unsaturated branched or straight chain alcohols, such as for example, decyl alcohol, lauryl alcohol, myristyl alcohol, cetyl alcohol, stearyl alcohol, oleyl alcohol, linoleyl alcohol and linolenyl alcohol, and mixtures thereof. Suitable alkoxylated alcohols include alkoxylated, typically ethoxylated, derivatives of (C10-C24) saturated or unsaturated branched or straight chain alcohols, more typically (C10-C20) saturated or unsaturated branched or straight chain alcohols, which may include, on average, from 1 to 22 alkoxyl units per molecule of alkoxylated alcohol, such as, for example, ethoxylated lauryl alcohol having an average of 5 ethylene oxide units per molecule. Mixtures of these alkoxylated alcohols may be used. Suitable fatty acids include (C10-C24) saturated or unsaturated carboxylic acids, more typically (C10-C22) saturated or unsaturated carboxylic acids, such as, for example, lauric acid, oleic acid, stearic acid, myristic acid, cetearic acid, isostearic acid, linoleic acid, linolenic acid, ricinoleic acid, elaidic acid, arichidonic acid, myristoleic acid, and palmitoleic acid, as well as neutralized versions thereof. Suitable fatty acid esters include esters of (C10-C24) saturated or unsaturated carboxylic acids, more typically (C10-C22) saturated or unsaturated carboxylic acids, for example, propylene glycol isostearate, propylene glycol oleate, glyceryl isostearate, and glyceryl oleate, and mixtures thereof. Suitable alkanolamides include aliphatic acid alkanolamides, such as cocamide MEA (coco monoethanolamide) and cocamide MIPA (coco monoisopropanolamide), as well as alkoxylated alkanolamides, and mixtures thereof.

Suitable Zwitterionic surfactants are known compounds. Any Zwitterionic surfactant that is acceptable for use in the intended end use application and is chemically stable at the required formulation pH is suitable as the optional Zwitterionic surfactant component of the composition of the present invention, including, for example, those which can be broadly described as derivatives of aliphatic quaternary ammonium, phosphonium, and sulfonium compounds in which the aliphatic radicals can be straight chain or branched and wherein one of the aliphatic substituents contains from about 8 to about 24 carbon atoms and one contains an anionic water-solubilizing group such as carboxyl, sulfonate, sulfate, phosphate or phosphonate.

In an embodiment, the Zwitterionic surfactant includes one or more sulfobetaines (INCI: sultaines). In an embodiment, the one or more sulfobetaines are selected from the sulfobetaines of Formula VI; the amido sulfobetaines of Formula VII; and combinations thereof:

R¹—N⁺(CH₃)₂—CH₂CH(OH)CH₂SO₃— Formula VI

R¹—CO—NH—(CH₂)₃—N⁺(CH₃)₂—CH₂CH(OH)CH₂SO₃— Formula VII

wherein R¹ is a saturated or unsaturated C6-22 alkyl residue, preferably C8-18 alkyl residue, in particular a saturated C10-16 alkyl residue, for example a saturated C12-14 alkyl residue. In an embodiment, the one or more sulfobetaines are selected from (designated in accordance with INCI): Cocamidopropyl Hydroxysultaine, Coco Hydroxysultaine, Coco Sultaine, Erucamidopropyl Hydroxysultaine, Lauryl Hydroxysultaine, Lauryl Sultaine, Oleamidopropyl Hydroxysultaine, Tallow amidopropyl Hydroxysultaine, and combinations thereof.

The personal care composition according to the present disclosure may optionally further include other additional components, such as, for example, preservatives such as benzyl alcohol, methyl paraben, propyl paraben and imidazolidinyl urea, thickeners and viscosity modifiers such as block polymers of ethylene oxide and propylene oxide, electrolytes, such as sodium chloride, sodium sulfate, and polyvinyl alcohol, pH adjusting agents such as citric acid, succinic acid, phosphoric acid, sodium hydroxide, and sodium carbonate, perfumes, dyes, and sequestering agents, such as disodium ethylenediamine tetra-acetate.

The personal care composition of the present disclosure is used in a manner known in the art, for example, in the case of a cleanser or shampoo, by application of the cleanser or shampoo to the skin and/or hair and optionally rinsing the cleanser or shampoo off of the skin and/or hair with water.

Home Care or Industrial Cleaning

In one embodiment, the present disclosure is directed to a home care or industrial cleaning composition, such as a liquid detergent, a laundry detergent, a hard surface cleanser, a dish wash liquid, or a toilet bowl cleaner, that includes one or more potassium-containing amphoacetate and/or betaine surfactants, water, and one or more additives as described herein. Such cleaning compositions may optionally further include one or more of water miscible organic solvents, such as alcohols and glycols, and/or one or more additives.

Suitable additives are known in the art and include, for example, organic builders, such as organophosphonates, inorganic builders, such as ammonium polyphosphates, alkali metal pyrophosphates, zeolites, silicates, alkali metal borates, and alkali metal carbonates, bleaching agents, such as perborates, percarbonates, and hypochlorates, sequestering agents and anti-scale agents, such as citric acid and ethylenediaminetetraacetic acid, inorganic acids, such as phosphoric acid and hydrochloric acid, organic acids, such as acetic acid, abrasives, such as silica or calcium carbonate, antibacterial agents or disinfectants, such as triclosan and cationic biocides, for example (N-alkyl)benzyldimethylammonium chlorides, fungicides, enzymes, opacifing agents, pH modifiers, dyes, fragrances, and preservatives.

In an embodiment the home care or industrial cleaning composition for cleaning fabrics or hard surfaces includes one or more potassium-containing amphoacetate and/or betaine surfactants, water, and a home care or industrial cleaner benefit agent.

In an embodiment the home care or industrial cleaner benefit agent is selected from soil release agents, fabric softener, surfactants, builders, binders, bleach and fragrances.

The present disclosure also provides a method for cleaning a substrate selected from hard surfaces and fabrics by applying a composition of the present disclosure to the substrate.

Oilfield

The potassium-containing amphoacetate and/or betaine surfactants of the present disclosure are also useful as clay stabilizing agents in aqueous fluid compositions used in oilfield applications. As used herein, the term “clay stabilizing formulation” refers to a composition which is capable of delivering a clay stabilizing agent into a subterranean formation. For instance, the clay stabilizing formulation may be utilized with a separate drilling fluid, drill-in fluid, completion fluid, stimulation fluid, fracturing fluid, acidizing fluid, remedial fluid, scale inhibition fluid, circulating fluid, gravel pack fluid or the like or may itself be any of the aforementioned fluids. Such fluids may contain a gelling agent to increase the viscosity of the fluid. The clay stabilizing agent can also be utilized in cementing fluids such as a cement slurry or a cement spacer, in certain illustrative embodiments. In a preferred embodiment, the clay stabilizing agent is entrained within the aqueous fluid. In other embodiments, the clay stabilizing agent can be made available as a solid material without being dissolved or entrained in the aqueous fluid. In certain embodiments, the clay stabilizing formulation exhibits an acidic, alkaline or neutral pH, such as those in the range of from about 1 to 11, and may be utilized with aqueous fluids having an acidic, alkaline or neutral pH.

When combined with an aqueous fluid to render an aqueous clay stabilizing formulation, the clay stabilizing agent is capable of reducing or substantially eliminating damage to the formation caused by the clay subterranean materials. The presence of the clay stabilizing agent eliminates or reduces the tendency of the clay subterranean materials to swell or disintegrated/migrate upon contact with the clay stabilizing formulation.

The phrase “subterranean formation” encompasses both areas below exposed earth and areas below earth covered by water, such as an ocean or fresh water.

The term “clay subterranean materials” includes sand and/or clays which swell, disperse, disintegrate or otherwise become disrupted, thereby demonstrating an increase in bulk volume, in the presence of foreign aqueous well treatment fluids, such as drilling fluids, stimulation fluids, gravel packing fluids, etc. The term also includes those sand and/or clays which disperse, disintegrate or otherwise become disrupted without actual swelling. For example, clays which, in the presence of foreign aqueous well treatment fluids, expand and may be disrupted by becoming unconsolidated, thereby producing particles that migrate into a borehole shall also be included by the term.

Clay subterranean materials which may be effectively treated with the clay stabilizer formulation may be of varying shapes, such as, for example, minute, plate-like, tube-like and/or fiber-like particles having an extremely large surface area. Examples include clay minerals of the montmorillonite (smectite) group such as montmorillonite, saponite, nontronite, hectorite and sauconite, the kaolin group such as kaolinite, nacrite, dickite, and halloysite, the hydrousmica group such as hydrobiotite, gluaconite, illite and bramallite, the chlorite group such as chlorite and chamosite, clay minerals not belonging to the above group such as vermiculite, attapulgite and sepiolite and mixed-layer varieties of such clay minerals and groups. Other mineral components may be further associated with the clay.

In certain embodiments, there is provided a method of inhibiting the swelling and/or migration of clay subterranean materials during drilling. The method includes drilling a borehole with a drilling fluid that includes a clay stabilizing agent selected from potassium-containing amphoacetate and/or betaine surfactants of the present disclosure.

In other embodiments, there is provided a method of inhibiting the swelling and/or migration of clay subterranean materials encountered during the drilling of a subterranean formation. The method includes circulating in the subterranean formation a water-based well treatment fluid that includes an aqueous continuous phase and a clay stabilizing agent selected from potassium-containing amphoacetate and/or betaine surfactants of the present disclosure.

In additional embodiments, there is provided a method of inhibiting the swelling and/or migration of clay subterranean materials during production. The method includes circulating a production fluid through a borehole with a circulating fluid that includes a clay stabilizing agent selected from potassium-containing amphoacetate and/or betaine surfactants.

In certain embodiments, there is provided a method of extracting oil from an oil containing subterranean formation. The method includes providing through a first borehole, a pressurized water-based well treatment fluid that includes an aqueous continuous phase and a clay stabilizing agent selected from potassium-containing amphoacetate and/or betaine surfactants of the present disclosure, and recovering oil from the subterranean formation through a second borehole. In certain embodiments, the subterranean formation was previously hydraulically fractured and oil was previously extracted.

Generally, the clay stabilizing agent is present in the clay stabilizing formulation in an amount sufficient to reduce either or both of surface hydration based swelling and/or osmotic based swelling of clay subterranean materials. The exact amount of the clay stabilizing agent present in a particular clay stabilizing formulation may be determined by a trial and error method of testing the combination of clay stabilizing formulation and clay formation encountered. In one embodiment, the amount of clay stabilizing agent of the present disclosure used in the clay stabilizing formulation ranges from about 0.25 gallons per thousand gallons to about 5 gallons per thousand gallons (“gpt”) of clay stabilizing formulation. In another embodiment, the amount of stabilizer in the clay stabilizing formulation is at least 0.5 gallons per thousand gallons. In still another embodiment, the amount of clay stabilizing agent present in the clay stabilizing formulation ranges from about 0.05% to about 0.5% by volume of the clay stabilizing formulation.

In another embodiment, the clay stabilizing formulation optionally contains one or more conventional additives. Examples of such additives include, but are not limited to, gelling materials, thinners, fluid loss control agents, encapsulating agents, bactericides, gel breakers, foaming agents, irom control agents, stabilizers, lubricants, penetration rate enhancers, defoamers, corrosion inhibitors, lost circulation fluids, anti-bit balling agents, neutralizing agents, pH buffering agents, surfactants, proppants, and sand for gravel packing.

Examples of gelling materials include, but are not limited to, bentonite, sepiolite clay, attapulgite clay, anionic high-molecular weight polymers and biopolymers.

Examples of thinners include, but are not limited to, lignosulfates, modified lignosulfates, polyphosphates, tannins, and low molecular weight polyacrylates.

Examples of fluid loss control agents include, but are not limited to, synthetic organic polymers, biopolymers and mixtures thereof, modified lignite polymers, modified starches and modified celluloses.

Examples of encapsulating agents include, but are not limited to, synthetic materials, organic materials, inorganic materials, biopolymers or mixtures thereof. The encapsulating agent may be anionic, cationic or non-ionic in nature.

While specific embodiments are discussed, the specification is illustrative only and not restrictive. Many variations of this disclosure will become apparent to those skilled in the art upon review of this specification.

The present disclosure will further be described by reference to the following examples. The following examples are merely illustrative and are not intended to be limiting.

### Example 1

Preparation of Potassium Lauroamphoacetate (30.69% Actives)

Water (431.3 g) and 45% potassium hydroxide (45% KOH) (48.65 g) were added to a 4-necked 2 liter (“L”) resin kettle with bottom valve equipped with a stirrer, thermometer, pH probe, and two addition funnels. Lauro imidazoline (218.07 g), which had been pre-melted at a temperature of 65° C. to 70° C., was then added to the reaction kettle. The temperature was raised to 79° C.-82° C. After the temperature reached 79° C.-82° C., the reaction mixture was held one hour.

After a 1 hour hold, 44.28 grams of 80% Monochloroacetic Acid (80% MCA) was added to the reaction mixture. After this addition, the 20% aqueous pH of the reaction mixture was 8.5-9.5. With the addition funnels, a co-feed of 66.55 grams 80% MCA was added with 128.86 grams 45% KOH, at a rate to keep the reaction temperature between 79-82° C., and the 20% aqueous pH between 8.5 and 9.5. After the completion of the co-feed, the MCA addition funnel was rinsed with 16.6 grams of water. The reaction mass was held for four hours between 79-82° C. while the pH was maintained at 8.5-9.5 with the as-needed dropwise addition of 45.67 grams 45% KOH. The reaction was deemed complete when the residual amidoamine level was less than 1% and the residual SMCA level was less than 125 ppm.

The product was adjusted to a level of 40.7% solids with water. The pH of a 20% aqueous solution was 9.21. The product further analyzed 1.3% potassium glycolate, 8.71% potassium chloride, actives at 30.69% (actives calculation is Actives=Solids−Potassium Chloride−Potassium Glycolate) and a neat viscosity of 48 cps at 25° C.

### Example 2

Preparation of Potassium Lauroamphoacetate (41.09% Actives)

Water (307.12 g) and 45% potassium hydroxide (45% KOH) (59.27 g) were added to a 4-necked 2 L resin kettle with bottom valve equipped with a stirrer, thermometer, pH probe, and two addition funnels. Lauroimidazoline (265.69 g), which had been pre-melted at a temperature of 65° C. to 70° C., was then added to the reaction kettle. The temperature was raised to 79° C.-82° C. After the temperature reached 79° C.-82° C., the reaction mixture was held one hour.

After a 1 hour hold, 53.95 grams of 80% Monochloroacetic Acid (80% MCA) was added to the reaction mixture. After this addition, the 20% aqueous pH of the reaction mixture was 8.5-9.5. With the addition funnels, a co-feed of 81.09 grams 80% MCA was added with 128.86 grams 45% KOH, at a rate to keep the reaction temperature between 79-82° C., and the 20% aqueous pH between 8.5 and 9.5. After the completion of the co-feed, the MCA addition funnel was rinsed with 20.23 grams of water. The reaction mass was held for four hours between 79-82° C. while the pH was maintained at 8.5-9.5 with the as-needed dropwise addition of 55.64 grams 45% KOH. The reaction was deemed complete when the residual amidoamine level was less than 1% and the residual SMCA level was less than 125 ppm.

The product was adjusted to a level of 49.6% solids with water. The pH of a 20% aqueous solution was 9.30. The product further analyzed 1.3% potassium glycolate, 7.21% Potassium chloride, actives at 41.09% (actives calculation is Actives=Solids−Potassium Chloride−Potassium Glycolate) and a neat viscosity of 3,400 cps at 25° C.

### Example 3

Preparation of Potassium Cocoamphoacetate (31.02% Actives)

Water (441.41 g) and 45% potassium hydroxide (45% KOH) (42.96 g) were added to a 4-necked 2 L resin kettle with bottom valve equipped with a stirrer, thermometer, pH probe, and two addition funnels. Cocoimidazoline (217.76 g), which had been pre-melted at a temperature of 65° C. to 70° C., was then added to the reaction kettle. The temperature was raised to 79° C.-82° C. After the temperature reached 79° C.-82° C., the reaction mixture was held one hour.

After a 1 hour hold, 42.76 grams of 80% Monochloroacetic Acid (80% MCA) was added to the reaction mixture. After this addition, the 20% aqueous pH of the reaction mixture was 8.5-9.5. With the addition funnels, a co-feed of 64.26 grams 80% MCA was added with 128.68 grams 45% KOH, at a rate to keep the reaction temperature between 79-82° C., and the 20% aqueous pH between 8.5 and 9.5. After the completion of the co-feed, the MCA addition funnel was rinsed with 16.58 grams of water. The reaction mass was held for four hours between 79-82° C. while the pH was maintained at 8.5-9.5 with the as-needed dropwise addition of 45.60 grams 45% KOH. The reaction was deemed complete when the residual amidoamine level was less than 1% and the residual SMCA level was less than 125 ppm.

The product was adjusted to a level of 40.1% solids with water. The pH of a 20% aqueous solution was 9.03. The product further analyzed 1.24% potassium glycolate, 7.85% Potassium chloride, actives at 31.02% (actives calculation is Actives=Solids−Potassium Chloride−Potassium Glycolate) and a neat viscosity of 12 cps at 25° C.

### Example 4

Preparation of Potassium Cocoamphoacetate (40.49% Actives)

Water (292.55 g) and 45% potassium hydroxide (45% KOH) (54.41 g) were added to a 4-necked 2 L resin kettle with bottom valve equipped with a stirrer, thermometer, pH probe, and two addition funnels. Cocoimidazoline (275.79 g), which had been pre-melted at a temperature of 65° C. to 70° C., was then added to the reaction kettle. The temperature was raised to 79° C.-82° C. After the temperature reached 79° C.-82° C., the reaction mixture was held one hour.

After a 1 hour hold, 54.15 grams of 80% Monochloroacetic Acid (80% MCA) was added to the reaction mixture. After this addition, the 20% aqueous pH of the reaction mixture was 8.5-9.5. With the addition funnels, a co-feed of 81.39 grams 80% MCA was added with 162.97 grams 45% KOH, at a rate to keep the reaction temperature between 79-82° C., and the 20% aqueous pH between 8.5 and 9.5. After the completion of the co-feed, the MCA addition funnel was rinsed with 21.00 grams of water. The reaction mass was held for four hours between 79-82° C. while the pH was maintained at 8.5-9.5 with the as-needed dropwise addition of 57.76 grams 45% KOH. The reaction was deemed complete when the residual amidoamine level was less than 1% and the residual SMCA level was less than 125 ppm.

The product was adjusted to a level of 50.8% solids with water. The pH of a 20% aqueous solution was 9.20. The product further analyzed 1.91% potassium glycolate, 8.40% Potassium chloride, actives at 40.49% (actives calculation is Actives=Solids−Potassium Chloride−Potassium Glycolate) and a neat viscosity of 5,000 cps at 25° C.

### Example 5

Body Wash (Potassium Lauroamphoacetate)

Water and sodium cocoyl glycinate (36.36%/wt) were added to a beaker. This mixture was heated to 65° C., and lauric acid (1%/wt) was added and mixed until it was completely melted into the batch. Then, potassium lauroamphoacetate (25%/wt) was added to the batch and mixed for 15 minutes. The heat was turned off and batch cooled to 25° C. At 40° C., cocamidopropyl hydroxysultaine (25%/wt) was added to the batch and the pH of the formulation was adjusted to 7.5 with 50% citric acid solution. Phenoxyethanol and fragrance were added to the formulation at 0.5% each for preservation and aesthetic appeal of the formulation.

### Example 6

Body Wash (Potassium Cocoamphoacetate)

Water and sodium cocoyl glycinate (36.36%/wt) were added to a beaker. This mixture was heated to 65° C., and lauric acid (1%/wt) was added and mixed until it was completely melted into the batch. Then, potassium cocoamphoacetate (25%/wt) was added to the batch and mixed for 15 minutes. The heat was turned off and batch cooled to 25° C. At 40° C., cocamidopropyl hydroxysultaine (25%/wt) was added to the batch and the pH of the formulation was adjusted to 7.5 with 50% citric acid solution. Phenoxyethanol and fragrance were added to the formulation at 0.5% each for preservation and aesthetic appeal of the formulation.

### Comparative Example 7

Body Wash (Sodium Lauroamphoacetate)

Water and sodium cocoyl glycinate (36.36%/wt) were added to a beaker. This mixture was heated to 65° C., and lauric acid (1%/wt) was added and mixed until it was completely melted into the batch. Then, sodium lauroamphoacetate (25%/wt) was added to the batch and mixed for 15 minutes. The heat was turned off and batch cooled to 25° C. At 40° C., cocamidopropyl hydroxysultaine (25%/wt) was added to the batch and the pH of the formulation was adjusted to 7.5 with 50% citric acid solution. Phenoxyethanol and fragrance were added to the formulation at 0.5% each for preservation and aesthetic appeal of the formulation.

### Example 8

Foam Performance

The body washes from Examples 5-7 were compared for foam performance specifically foam height at 30 seconds using a Kruss DFA dynamic 100 foam analyzer.

A 1% solution of each body wash was prepared. 50 ml 1% solution was accurately weighed and added to a glass cylinder in the DFA foam analyzer. Air was pumped through the cylinder for 25 seconds at a flow rate of 0.3 L/min. The foam height was measured at 30 seconds for each of the three body wash formulations. It was observed that the initial foam height of the formulation with potassium cocoamphoacetate (192 mm) was slightly higher than that of the control formulation made with sodium lauroamphoacetate (184 mm), which was an unexpected result.

### Example 9

Blend (Potassium Lauroamphoacetate)

Water and sodium cocoyl methyl taurate (28%/wt) were added to a beaker and the batch was heated to 65° C. Potassium lauroamphoacetate (30%/wt) was added to the batch and mixed for 20-25 minutes. When batch reached 65° C., sodium cocoyl isethionate (40%/wt) was added to the batch and mixed until it was completely melted into the blend. The heat was then turned off and batch was cooled to 25° C. The pH of the blend was adjusted to 10.5 with 50% sodium hydroxide solution. The blend was clear in appearance and initial viscosity was measured at 900 cps (LV #4 @ 10 rpm).

### Comparative Example 10

Blend (Sodium Lauroamphoacetate)

Water and sodium cocoyl methyl taurate (28%/wt) were added to a beaker and the batch was heated to 65° C. Sodium lauroamphoacetate (30%/wt) was added to the batch and mixed for 20-25 minutes. When batch reached 65° C., sodium cocoyl isethionate (40%/wt) was added to the batch and mixed until it was completely melted into the blend. The heat was then turned off and batch was cooled to 25° C. The pH of the blend was adjusted to 10.5 with 50% sodium hydroxide solution. The blend was clear in appearance and initial viscosity was measured at 750 cps (LV #4 @ 10 rpm).

### Example 11

Stability

Samples of both blend examples were aged in ovens at 4° C., 25° C. and 45° C. respectively for 13 weeks. Aging studies conducted on the blend with sodium lauroamphoacetate demonstrated physical instability, the samples turned white on aging and there was also a significant viscosity build in aged samples. The initial viscosity of the samples was recorded at 750 cps whereas the sample aged at 45° C. at 13 weeks was measured at 15000 cps.

In contrast, there were unexpected results with the blend made with potassium lauroamphoacetate. The samples remained clear even after aging at 45° C. for 13 weeks. There was a viscosity increase noted but this was not as high as the blend with sodium lauroamphoacetate. The viscosity of the sample was 10000 cps. The blend formulation with potassium lauroamphoacetate exhibited a better stability profile than the sodium lauroamphoacetate.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition. If there is any conflict in the usages of a word or term in this specification and one or more patent or other documents that may be incorporated herein by reference, the definitions that are consistent with this specification should be adopted.

We claim:

1. A composition comprising at least one potassium-containing amphoacetate and/or betaine surfactant, water and at least one additional surfactant selected from the group consisting of anionic surfactants, cationic surfactants, non-ionic surfactants, zwitterionic surfactants, and mixtures thereof.

2. The composition of claim 1 comprising at least one potassium-containing amphoacetate selected from the group consisting of:
a) amphoacetates according to Formula I: 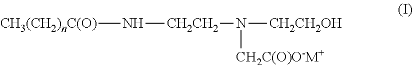
wherein n is from 6 to 20 and M is a potassium cation;
b) amphodiacetates according to Formula II: 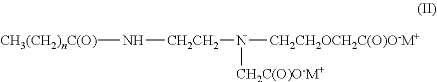
wherein n is from 6 to 20 and M is a potassium cation;
c) amphodiacetates according to Formula III: 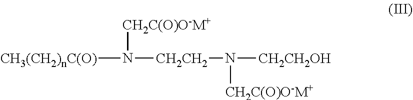
wherein n is from 6 to 20 and M is a potassium cation;
d) amphodiacetates according to Formula IV: 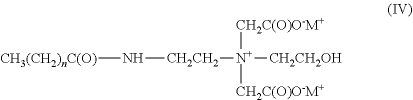
wherein n is from 6 to 20 and M is a potassium cation; and combinations thereof.

3. The composition of claim 1 comprising at least one potassium-containing betaine comprising potassium chloride and a betaine selected from the group consisting of coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and combinations thereof.

4. The composition of claim 1, wherein the at least one additional surfactant is an anionic surfactant.

5. The composition of claim 4 further comprising a non-ionic surfactant.

6. The composition of claim 1 further comprising at least one additional benefit agent selected from the group consisting of emollients, moisturizers, conditioners, skin conditioners, hair conditioners, vitamins, antioxidants, free-radical scavengers, abrasives, anti-UV agents, UV absorbers, antimicrobial agents, antibacterial agents, antifungal agents, anti-acne agents, antiseborrhoeic agents, anti-ageing agents, anti-wrinkle agents, anti-inflammatory agents, refreshing agents, cicatrizing agents, vascular-protection agents, antiperspirants, deodorants, immunomodulators, nourishing agents, essential oils, fragrances, and combinations thereof.

7. The composition of claim 1 further comprising one or more water miscible organic solvents.

8. The composition of claim 1 further comprising at least one additive selected from the group consisting of organic builders, inorganic builders, bleaching agents, sequestering agents, anti-scale agents, inorganic acids, organic acids, abrasives, antibacterial agents, disinfectants, fungicides, enzymes, opacifing agents, pH modifiers, dyes, and combinations thereof.
